(19) United States
(12) Patent Application Publication
(10) Pub. No.: US 2003/0143283 A1
(43) Pub. Date: Jul. 31, 2003

(54) Bone repair material

(76) Inventor: Andrew Tofe, Lakewood, CO (US)

Correspondence Address: Glenn K. Beaton, Gibson, Dunn & Crutcher LLP, Suite 4100, 1801 California Street, Denver, CO, 80202, US

(21) Appl. No.: 10/057,796

(22) Filed: Jan. 28, 2002

(51) Int. Cl. A61K 38/18; A61K 9/14; A61K 35/32

(52) U.S. Cl. 424/549; 514/12

(57) ABSTRACT

A method and composition for repairing bone in a human. A high-molecular hydrogel such as a hyaluronate is mixed with a bovine or other animal bone particulate or with artificial bone such as hydroxyapatite particulate. A growth-inducing peptide may also be included.

### FIELD OF THE INVENTION

This invention relates to the field of surgical repair of skeletal elements. More specifically, the invention is a method and composition for filling or otherwise repairing bone damage using a material having a paste-like consistency and comprised of non-human bone in a suitable granularity suspended in a biocompatible gel.

### BACKGROUND OF THE INVENTION

Medicine often involves repairing bone that is damaged by disease, trauma, osseous surgery or other mechanisms. Sometimes this can be accomplished merely by relocated disrupted elements into natural proximity and fixing them in place until they can heal together. This is the approach taken in repairing ordinary limb fractures, for example. The fractured bone is re-set, then immobilized for a period of weeks in a rigid or semi-rigid cast or splint as the fractured elements regrow.

Sometimes, however, this approach is insufficient because the patient has lost some of the bone. This can happen in certain kinds of trauma where the bone is so badly shattered that it cannot feasibly be pieced together. More often, it happens as a result of disease that destroys bone mass or as the result of osseous surgery in which destruction of bone mass is unavoidable. In these cases, there is no “piece” of patient bone to re-set into proper position for healing and regrowth. Instead, there is a void that must somehow be filled. The filling of this void requires a material that is not only biocompatible but preferably will accept in-growing natural bone as the site heals. In that manner, the material ideally will eventually become an integral part of the skeletal structure.

Numerous such materials have been employed with varying degrees of success. One approach is to use bone material recovered from the patient himself, or so-called autologous bone. That approach is advantageous in that it avoids biocompatibility problems and bio-rejection problems. Also, the implanted material tends to be sticky so that it adheres to the implant site. However, such an approach necessarily involves two surgical procedures, two surgical sites and two healing processes: one for the original injury and a second for the site of the donated material. This means greater cost, and increased risk of infection and morbidity for a patient that is already seriously ill or injured. Also, such an approach can require a great deal of time and surgical skill as the surgeon removes the donated material from the donation site, shapes and fits it to the primary site, and then repairs both sites. Finally, there is quite obviously a limit to the amount of bone in the patient's body available to be sacrificed as donor material.

Another approach has been to use human bone that is not from the patient, called allograft bone. Allograft bone is harvested from cadavers. It contains bone morphogenic proteins (“BMP”) and is available sterile and demineralized. Such material becomes integrally incorporated into the patient's own skeletal system.

Allograft is typically offered by commercial medical suppliers in dry granulated or powdered form of varying fineness. These dry granules or powder lack sufficient cohesiveness and adhesion for filling an osseous void. Therefore, they are mixed with an appropriate carrier. The carrier in the past has sometimes been the patient's own blood. Such a carrier is of course plentiful at the surgical site, is biocompatible with the patient, and contains BMPs to promote new growth in the allograft bone elements suspended in it. On the other hand, using the patient's own blood necessitates a mixing step which might not be controlled precisely in the operating room to achieve the desired consistency. When the blood used is not from the patient but is donated blood, issues of type compatibility, disease transmission and varying quality are presented. Whether the blood is the patient's own or is donated, blood is not of the ideal consistency or viscosity for such an application.

Allograft bone in combination with an artificial gel is sold under the trademark GRAFTON,® by Osteotech, Inc. Grafton is a mixture of glycerol and demineralized bone powder, and is described in U.S. Pat. No. 5,073,373. The glycerol in this product is suitable in consistency and viscosity for this application, but suffers from certain functional drawbacks. Because it is water soluble, it can dissolve too quickly and allow dispersement of the suspended bone after being placed. It also tends to melt, or at least become less viscous, at body temperature. This interferes with properly placing the material in the desired site. Attempts to perfect combinations of allograft bone and glycerol or other gels are described in U.S. Pat. Nos. 5,290,558; 5,314,476; 5,507,813; 4,172,128 and 4,191,747.

As a substitute for glycerol, there have been attempts at using either human or animal (such as bovine) collagen in combination with demineralized allograft bone. A drawback to collagen is its relatively slow rate of adsorption into the body. Because new natural bone cannot grow into the implanted material until the collagen carrier vacates the material, this slow rate of adsorption slows the healing and the development of skeletal structural strength. In the case of bovine or other animal collagen, there is also concern about the transmission of animal diseases to the patient.

U.S. Pat. No. 6,030,635 by Gertzman discloses a malleable bone putty which includes allograft bone powder suspended in a high-molecular weight hydrogel, preferably a hyaluronate. Hyaluronon is a polysaccharide that occurs naturally in the body in the form of hyaluronic acid or in the salt form such as sodium hyaluronate. It is highly hydrophilic and extremely lubricous. The data presented in the Gertzman patent suggest that the high-molecular weight hydrogel allows for small particle size in the allograft material. The contents of such patent are hereby incorporated by reference.

### SUMMARY OF THE INVENTION

The present invention is a method and composition for bone repair using non-human bone materials such as granulated or powdered bovine or other animal bone, suspended in a hydrogel. In a preferred embodiment, the hydrogel includes hyaluronate or some other high-molecular weight biocompatible gel-like material.

In a particularly preferred embodiment, the composition includes a growth-inducing peptide. In an alternative embodiment, the composition is synthetic bone such as hydroxyapatite, again preferably but not necessarily with a growth-inducing peptide.

### DETAILED DESCRIPTION OF THE INVENTION

The composition of the present invention uses a synthetic or animal bone material. In synthetic form, it may be well-known artificial bone material such as hydroxyapatite. In the animal form, it may be bovine bone. In either case, the material is preferably ground or milled to a suitable small-sized grain or to a powder. The process of manufacturing bone implant material from artificial compositions such as hydroxyapatite or from animal bone is well-known and not further described here.

The prepared synthetic or animal bone material is suspended in a carrier. The carrier in the preferred embodiment is a high-molecular weight hydrogel. Especially useful is hyaluronate due to its high molecular weight and hydrophilic properties. Methods of preparing a hyaluronate hydrogel with allograft material is described in the Gertzman patent mentioned above, incorporated herein by reference, and may also be used in the present invention.

To promote bone growth, the composition in a preferred embodiment may also include a peptide. Preferred peptides are described in U.S. Pat. Nos. 5,635,482; 5,958,428; and 5,354,736, all by Bhatnagar, the contents of which are hereby incorporated by reference.

What is claimed is:

1. A composition for application to a bone defect site to promote new bone growth, comprising a mixture of non-human bone-like powder; a hydrogel carrier; and a growth-inducing peptide.
